(19) United States
(12) Patent Application Publication
(10) Pub. No.: US 2022/0068156 A1
(43) Pub. Date: Mar. 3, 2022

(54) Re-Designed Diagnostic Process for Psychiatry/Psychoanalysis

(71) Applicant: Shelley Baum-Brunner, Philadelphia, PA (US)

(72) Inventor: Shelley Baum-Brunner, Philadelphia, PA (US)

(21) Appl. No.: 16/873,974

(22) Filed: Sep. 2, 2020

(51) Int. Cl. G09B 19/00

(52) U.S. Cl. CPC G09B 19/00

(57) ABSTRACT

The object of the patent is to change the diagnostic proceedings in the fields of psychiatry/psychoanalysis since this ABSTRACT and the “Dishonoring and Honoring Freud” (exhibit A) show that despite huge accomplishments Freud made to the field of psychiatry, the process for diagnosing outlined by Freud is fraudulent. By saying ONLY FREUD can diagnose—according to his own writings—and once given a diagnosis, it can never be changed, the diagnostic procedure is more rooted in social/cultural needs than medical terms/medical science, done to camouflage the fact that the Prince of Austria and Freud compacted a fraudulent contract—to protect Freud, a JEW, in Nazi Austria from Nazi terror, and to give him more business clients in a waning practice of mainly female hysterics in exchange for a “safe” way for the prince to get a new wife.

As a catholic, the Prince could not divorce, but the Prince looked to Freud to diagnose his wife with such an ailment as “delusional disorder”/psychosis so that society would accept his shift to another partner, as his wife would be “medically” unable to perform her duties as mother, wife and princess and society would find this socially acceptable compared to a paramour alone or divorce.

In this way, Freud's pact with the Prince moved his up-to-then sound diagnoses (at one time in history w diagnoses like neurotic etc., his work was sound scientifically) to one that was medical nonsense twenty years later and perpetuated a fraud on the wife of the Prince, other wives of other friends of the prince (which helped improve Freud's practice from an economic standpoint), and on science itself, or on that part of Freud's science that up until then was sound.

By pronouncing that he would be the only one to be able to diagnose, Freud protected himself and the prince from being outed or found out. His word would be golden and no one else could debate it; each diagnosis could, he said, NEVER BE CHANGED. The prince and his new “wife” would be safe. It may also have been a psychological effect of feeling powerless in Austria against the ever-growing Nazi regime, and pronouncing himself Inviolable, as no other could do what he could do, a way to empower himself when he really felt powerless.

And though other diagnoses, like neurosis, etc were identified almost twenty years earlier and were scientifically based, or seem more accepted means of scientific analysis used, this diagnosis of delusional/psychotic had no basis in medical fact, but in social/political/economic need that met the needs of Freud in that time period and of the Prince, and their conjoined social needs constructed a fabricated false diagnosis. How medical a need is it if it meets the needs of a husband for a new wife, and that alone? Is the old wife Out of touch with reality? Or out of touch with how to protect herself when there is conspiracy to satisfy a husband's needs to have someone else be a new partner?

How much a medical reality could it possibly be when science bases itself on replication of results to show reliability, but another doctor was not allowed to look at the patient for diagnosing purposes, and that makes his diagnostic process run counter to science itself. Since according to Freud, other doctors could only treat and only treat for the diagnosis he created, there is a problem with the science at work here, since true science would accept growth and change from the treatments themselves, or should.

Since I argue that the medical diagnostic process Freud proposed years ago and is still used today!, is not medically sound or scientifically driven, it must be changed to one that is scientifically more reliable and verifiable. It has not changed in all these years based on a misunderstanding that it was needed to be done for science, but that was conflated with the real reason—personal cultural needs dictated how diagnosing was done then and done now, but since it was not science at all, that diagnostic process has to change now.

Specification: Changes to be Considered:

Rather than the same questions over and over driving the diagnosing (have you been diagnosed with this before? And affirmative answer leads to redo of that diagnosis), new questions need to be created (e.g. What is your key concern? How might I help you address that concern?? What things have helped you deal best with the area of concern? What least? If you were brought by a petitioner family member or neighbor or “friend”, what do THEY HAVE TO GAIN by your being here? In other words, dig deep as to what is really going on, more like an anthropologist, or a detective than a doc interested in filling another bed or saving one's own life).

There is a marked difference between an almost robotic question/answer that needs no real intelligence, no real analysis, no real observation, no real Scientific scrutiny and one where a doctor really digs deep, really thinks, really is creative with the questions and with answers that lead to something more than a pat, paint by numbers set. How ironic that smart doctors are reduced to rabbits in a cage and can do nothing more than repeat repeat repeat what has been done before. How does that honor the intelligence and training of a medical doctor??

And then aside from new questioning, also use other data sources (like the work already done with brain scans, so bio markers reinforce social questioning and reinforce more subjective information with more hard data). Since it is possible to see physical trauma in the brain, it would be possible to see trauma, and not, like Freud said, delusional disorder as an example; It could rule in and rule out other ideas. The Brain scans can reinforce or counter a more subjective finding. And remember too that perhaps at times—as happened with the Prince—it is psychiatry itself that is the abuser when they make calls for same diagnosis as before, since that is not what is needed, at all. And the brain scans will not know the source of the abuse and trauma but can see evidence of its existence or some other aspects being considered. It is an important upgrade since Freuds' day, and I honor it but make no claim that it is my work, only add commentary to how useful it can be.

In this way, with new questions and with more data sources, there is more dignity for the patient, less labeling in a harmful, abusive way, and more opportunities for insights to be addressed, not just repeating the past. In the same way, a worker with the Innocence Project in mind, would not ever say to a prisoner, because you were a prisoner before means you need to be here again, the new diagnosing, would also look for fresh avenues of data collection, and analysis, to see if there are ways to capture self-images in-tact, with less need to isolate someone, but to treat them with kindness and dignity, and therefore treat, not abuse them—and not do it the way Freud and the prince abused the prince's wife and the other wives of the princes' friends.

And the diagnosis of “psychotic/delusional” also needs to be struck from the DSM because it too, given what I've learned of its genesis, is pure fiction too and also therefore used for abuse. It served for the prince and for others as it can today, as a means to a. silence whistleblowers or victims of powerbrokers not wanting to lose hold on power even if corrupt b. to eviscerate voices in public outcry, news reports, civil or criminal courts, as “someone who cannot tell fact from fiction” who then would be deemed incompetent in a court of law to forward evidence faithfully or reliably. Though Freud tried to say the wives were seen as “unreliable,” yet in the Prince/Freud/Prince wife case, nothing could be further from the truth since we know the diagnostic process and that particular diagnosis is ITSELF FICTION, INTENDED to do exactly that—eviscerate that voice in court, to quash the prince's wife's objections to being sidelined as a mother and wife to make room for a “better” wife. The prince got Freud, vulnerable as he was as a JEW in Nazi Austria and vulnerable economically with being close to shutting down the hospital wards and clinics, to MISUSE SCIENCE to cover for something unethical the Prince wanted to do.

The object then is to forward another means for diagnosing, not dependent on only one voice but on many, on confirmation of others that the diagnosis is true, includes collaborative dialogue between patient and doctor to help make that determination as a way to confirm that the diagnosis is true, does not undercut a social need or be a voice for fraud. This new process will acknowledge that doctors do not speak with 100% authority, but as human voices with personal needs, and that cutting off the arrogance of “science” behind every medical pronouncement is a way to protect from the violation of science in order to better perpetuate good science, not fraud.

Given the misogyny involved with Freud/Prince pact, more time needs to be spent in diagnosing understanding what gets said, why it gets said, Under what circumstances, and seeing more context here and not assume what the doctor says is true. More skepticism of the doctor needs to be made here and practices that diagnose in three minutes (seen it done: What else were you diagnosed before/Ok you are that again) must be stopped, or diagnoses that favor men over women and what they want to see happen means we must look at possible ulterior motives of petitioners “concerned about their family members or friends” to try to forward science in the guise of science but for another aim—Something more untoward like personal gain.

In the same way a Prince had more power than his wife to get away with untruths, so too happens today even with our constitution, despite all the benefits afforded the individual. Those already in power hold more power and sway to undercut someone's voice if they have less power, but that does not make the more powerful necessarily more righteous or true. Just like with the Prince, he had more power but it was a case of might, not right. With that bit of history in mind, we cannot allow the handicaps in implementation of our constitution to hold science up for ransom, and use it for unscientific gains as the Prince of Austria did a hundred fifty years ago or so.

Each voice must count here: “black lives matter”, “women's voices matter”. We need a psychiatric model that each one's voice is just one voice, one vote, and one plaintiff or patient heard in court should be viewed as powerfully as the rich are because that is what democracy is about. Science cannot be used for blackmail here. So, the patient voice needs as much validity as the doctor, given what we know can happen/has happened/is still happening in the name of science when really, in some cases, it's merely fraud.

We need to protect science and use reliability and validity as part of the process, to keep the process in check and from being abused or psychiatry from being abusive, a tool for abuse, as did happen in these historical annals, and we say “no more.”

History of the Idea

I have written about 302 mental health mandated/forced hospitalizations for years. I have written about it as a form of abuse, in books, and articles, and documents to police and to FBI (I am a unpaid citizen commissioned officer reporting on public corruption, and see this as a form of public corruption and abuse), and see related abuses at the University of Pennsylvania who used their investigative rules for academic integrity that one officer called “so egregiously illegal they would have to go into their office with guns blazing” to stop that abuse. Part of what makes it so illegal, is that they would use illegal practices (break and enter without a search warrant, Entrap people or try to, file false evidence to bring on a false arrest etc.), to try to capture me for fraud with my dissertation (though I was cleared and there was none) but then tell the person nothing was going on (when there was) and to see a psychiatrist in a systemic gaslighting abuse which AMA calls the worst form of psychological abuse there is. And this is part of the investigation that they use! So with that, I became interested in the ways psychological abuses are used to feign for truth, for science, for investigations, but are nothing but abuses in too many cases, like the one with the Prince and Freud.

So, psychological states of affairs can be used for political reasons, not unlike was described with Freud and the PRINCE OF AUSTRIA.

At the University, I was investigated (and cleared by the FBI and police) of fraud related to my dissertation, and like above, was subject to endless bouts of psychological torture and abuse. The student that started this process, David Loewenberg, and a faculty member, Susan Lytle, a neighbor of his, were both identified by the FBI as the source of serious defamation against me, and the source of serious abuse, so bad the FBI called it the worst abuse they have ever witnessed when conjoined with how it affected my son!. Loewenberg was so deranged that he used endless deep pockets of money to hire people to torture me and my son, In Nazi-like tortures; he was a student of that period of history as he was a student in a Jewish day school and in a Seminary at the college level. The schools would be horrified to know he used that information to forward more torture; their belief was studying Nazi-ism was a way to stop it from happening again.

But, Loewenberg felt slighted by me because the unborn child of mine when I was his teacher was not his offspring and for that my son and I paid with a false fraud charge to be investigated by U of P and then treated with its attendant psychological tortures and attempts at 302 hospitalizations and diagnoses of “delusional” to say these things were not happening when they were—not unlike the kinds of chananigans happening with Freud and with the Prince.

I did have a three-day evaluation in NYC that showed me to have PTSD, not psychosis and it said calling me delusional would be a disservice and run counter to my needs. But efforts to use psychological diagnosing as a modern-day political prisoner phenomenon is what that doctor meant to eliminate, to forestall. He was talking “pure diagnoses” using brain scans in a more modern-day approach to diagnosing, one supported here by me (see earlier information).

The University of Pennsylvania, like the Prince, wanted to use a fictional diagnosis to fraudulently undercut the veracity and truth of evidence against these frauds, in criminal and civil cases in my effort to right these wrongs. I have articulated these abuses in court case filings and other writings and so will not elaborate any further on these things now.

But, I will say that with dozens of false 302 hospitalizations, I had a rare ethnographer, insider/outsider point of view to help validate that my findings are correct and do often perpetrate more abuse, not help. Although the hospitals will say there are protections against false 302, they occur in paper only—multiple diagnoses by various doctors in fact only repeat—as Dr. Freud asked for—each other's diagnoses and past diagnoses, and are not really independent evaluations at all.

In science, independent evaluation would be required and that would mean evaluations done outside each other's knowledge base or without conversation, but in fact, these “independent' evaluations occur simultaneously—two next to each other and in consult with one another and mirroring each other to save time and repeat what Dr. Freud said should occur. Like that, a diagnosis is done in 3 minutes, but that forwards more fraud.

With longer and different interviews (see section on specification), and with true independence of thought, good and rightful diagnoses could occur. But, it does not in fact occur ever in those hospital settings—at least not in the state of Pa, for reason explained below. They will cover for each other—“tell me what you want and I will write it that way” was heard more than once while I waited for a doctor to get off the phone, as just one example of “independent”, a non-variable for science here.

Some of the reason it is done this way in Philadelphia and Pennsylvania anyway, is that Philadelphia has more doctors and hospitals and pharmaceuticals than any other place in the county and there is a bloat of medical personnel and facilities, so without a way to fill the beds, hospitals will go out of business and doctors will not be paid. City tax coffers will lose too. So, with economic pressure (not unlike the pressures Freud faced in Austria), a deal was made with the mayor to take from university hospitals and medical schools tax free educational status in exchange for filling hospital beds, so much so that one doctor at Friends Hospital in Philadelphia said, the law allows “any petitioner to say anyone for any reason belongs in the hospital and will be granted a bed 100%! Without evidence or proof or medical need!!!!”

So, financial incentive is one reason misdiagnosing occurs at the same levels as with Freud this many years later. We also see other examples of political pressures as a reason for making patients mental health hospital prisoners—and we need only think of Watergate. Martha Mitchell, wife of Nixon's attorney general screamed at the Washington Post to investigate Nixon as a criminal and for that, she ended up hospitalized against her will for over a year by Nixon! It was not until Deep Throat, acting in response to what happened to her, went in silence to get the information to the POST that Nixon was finally undone. But to justify the hospitalization, they called her delusional and YET she WAS in touch with reality—just an ugly reality as far as Nixon was concerned and he used the hospital and diagnoses of “delusional” to try to protect himself from a fall from grace. It happens more often than people realize or know.

Because of these experiences, I was curious to know what Freud had said on the subject of diagnosing and of “delusional/psychotic” so I read his works. At first, I was surprised to see he wrote that no one other than he could diagnose, and at first surmised, he was just arrogant, and that no one else could be as good as he. I did see right away accurately, though I was initially wrong as to the reason why, that that would mean diagnoses would not change. Once an x diagnosis, that would stay with you, and I knew from the questions I got in the hospital that it just relied on your say-so to the question, “so what were you diagnosed before?” Past is not just the past that way but past is present and future too. What was a diagnosis will be a diagnosis and no hope for changes.

Then, I got to more understanding about the Fraud with the prince, when I had someone I know look for famous people in Freud's practice, came up with the prince and then had him read the Prince's biography, looking for what kind of patients he had at the time, though we already knew by then from other sources that he had rich clientele. It was the prince biography that outlined the “delusional” diagnosis as a way for the prince to get the wife he wanted and that he told his friends about the “miracles of science”, and Freud's business grew. From that I surmised that delusional diagnosis happened later than the other diagnoses, and I was correct. From that information, I used the word Freud fraud—though honor him in other ways at other times for the work he did within science's bounds.

1. I am claiming that Freud's work on diagnosing pre-Nazi period were met with scientific rigor and were not part of my claim, but part of the work of his own accomplishments, and own work.
What is mine is the idea that 1. Diagnosing today suffers because it is repeated ad nauseum from what you have had before and can never be changed and that comes from Freud's dictates 2. That women in particular run the risk of having a “delusional” diagnosis though “delusional” is a fiction, met for social and economic reasons in a deal between Freud and the Prince of Austria 3. That only Freud (or an MD today, I would extrapolate) can do a diagnosis and it can never change 4. That these historical issues related to the Prince and Freud means that today part of psychiatry is rooted in Fraud not Freud and needs to be changed. 5. Because diagnosing under the order of no one else can do it or can never be changed, also has attached to it, an arrogance, a doctor knows best, a do not question, so with that arrogance a doctor can quickly determine outcome etc. of a diagnosis but really since it was pre-ordained, from before, so it acts as more fraud perpetrated today from the earlier stances that involved Fraud.
So, in my claims, CHANGE MEANS a. slower evaluations, with more time and more voices, including the patient's voice need also to be used b. An understanding that a negative evaluation could benefit a family member or other who is complaining, and should not to be taken seriously without further digging of what is said, why it is said, and how it benefits the petitioner and if that is just and scientific for the patient/defendant c. that “delusional” was done for reasons other than medical, so does not have the same clout as the work done earlier like when “neurotic” was found; it is fiction and is an indication of an underlying fraud and so should be discontinued. d. that doctor knows best is also a fiction at least to some extent, and needs to be modified so that a patient's voice and other voices get heard in this process too.
Thus,
1. We also know that diagnosing is a human endeavor, rife with biases, and personal pulls and gains, with trades and deals and what is good for the one (the prince) is not good for another (the wife). A diagnosis should never be read as a pure and unshakable truth. Never.
2. That psychosis as we know it does not exist as a scientific construct, but a social and economic one, and we must be wary of that diagnosis moving forward and be wary of how a person making the diagnosis can be influenced by more than medical needs.
In that vein, Freud not trusting other doctors to be good enough to do such a difficult medical procedure as diagnosing is now interpreted to mean that no other doctor could be trusted not to find him and the prince out, to root them out. And thus, the Self-Importance of the importance of the process itself needs to be taken down a few notches. Freud's words are not to be seen as “gold,” at least not after his relationship with the prince changed the course of actions in the psychiatric fields. Medical and social-political-economic must be viewed moving ahead as part of the possibility of doing diagnoses and why the diagnoses should not change the outcome of a court hearing, or an ability to hear the voices of complainants or defendants.
That what occurred opened up the door for Abuse, for the field of psychiatry to be not a healer but a vehicle for abuse, particularly for women, in a wall of words, a pretense as bold as the diagnosis itself, in ability to tell fact from fiction, reality from delusion. It was the diagnosis itself that was delusional, a hoax, a poseur, that ruined those women's lives and for hundreds of years other lives to follow. I know. I am one.
It is important to note that Freud, like any human being, is situated in a social context—when he lived, where he lived, social mores and beliefs at the time.
It would be terribly unrealistic and unfair to him to expect him to have been a women's libber, a gender neutral, modern thinking man devoid of gender bias, of misogyny even, at the time, given when, where he lived.
That plays in his favor. He is only human. But it is an important lesson almost a hundred fifty years later that we should not follow his scripts, his writing, in soldier-like fashion, mirror-imaging ways. Rather, we need understand that a JEW in Austria, ruled by Austrian authority/royalty, was motivated by more than pure science.
Even Wikipedia discusses his positive legacy on our current thought with the ideas of his like “free associations, transference, repression, unconscious ego and super ego, libido, death drive and neurotic drive.”
Nonetheless, it goes on to say that Freud's work “continues to generate extensive and highly contested debate with regard to its therapeutic efficacy, its scientific status, and whether it advances or is detrimental to the feminist cause.”
Clearly my insights and original work shows SERIOUS detriment to the feminist cause, at least in light of this one diagnosis, an afterthought of his other diagnoses, and one better to have not been thought of at all-delusional—a word to be stricken from the annals of psychiatric literature, from courtroom records and from the DSM.
Summary of New Process
1. There is an irony that for all the arrogance medical doctors sometimes show about how smart they are, Freud's process for diagnosing, reduced them to robot status (were you diagnosed this before? Well, then we do that again). The irony is that the further in stature away from medical doctors (psychiatrist, psychologist, therapist, art therapist etc), the more willing those docs were to trying on new ideas and new diagnostic questions and procedures. We hope that with new process and issues outlined, that more docs, more medical personnel, are willing to try more ways of doing things, now that we know Freud's way, for this aspect anyway, did not work.
2. In sum: There should be a new interview (new set of interview questions outlined earlier in specification section), new perspective (act more like an anthropologist who explains the normalcy of odd behavior in the culture of the individual), do brain scans (so physical evidence not just human subjective and there are probably patents for that item being done—see chart below of traditional vs. more enlightened diagnosing), new mantra (past is not present), new diagnoses lead to different treatments—more than medicine therapy, but to all kinds of modalities—talk, cognitive, mindfulness, art therapy, etc. This should all be viewed with the appendix A in mind of “Honoring/Dishonoring Freud.” I actually believe that Freud's construct of “dummy down” to doctors also influence the treatment. If they could not THINK to change diagnosis, they could not THINK to treat in any way other than what FREUD said, and so again, their own intelligence is slighted and medication—yes, works to some extent in some circumstances but comes with serious side effects, and toxicity—is a paint by numbers fix. Were doctors free to use their intelligence to diagnose (as should be) and free to use intelligence to think about ways to address concerns in treatment, doctors would use more than medicines to help treat patients, and involve themselves and their patients in more Modalities of treatment that “less statured” therapists are willing to try. What I am claiming here is that Freud for all the reasons outlined that he made the field think he was being scientific but was not, HANDICAPPED doctors too, not just the process and not just the patients. Somehow, given the history, the more powerful in stature (medical doctors vs social workers say), the less power they actually have since they are following fraudulent dictates of Freud that keep their power more limited than it otherwise could or should be.
3. People who bring claims of problems against others need to be scrutinized for what it can benefit to them (e.g getting them money or a new wife), and then diagnosing doctors need to be skeptical of them and skeptical that the need to fill beds or help a selfish husband is not a scientific reason for giving debilitating diagnoses. We are looking for therapy not abuse, and looking to avoid what happened to the women of Freud's day.
4. There is to be no delusional/psychotic diagnosis and diagnosing is not to be done as Freud described/prescribed, for that is fraud, and not science.
